(19) United States
(12) Patent Application Publication
(10) Pub. No.: US 2023/0397930 A1
(43) Pub. Date: Dec. 14, 2023

(54) BALLOON GUIDE CATHETER FOR RADIAL ACCESS

(71) Applicant: Crossroads Neurovascular, Prospect, KY (US)

(72) Inventor: Mahan Ghiassi, Prospect, KY (US)

(73) Assignee: Crossroads Neurovascular, Prospect, KY (US)

(21) Appl. No.: 18/180,226

(22) Filed: Mar. 8, 2023

Related U.S. Application Data
(60) Provisional application No. 63/351,137, filed on Jun. 10, 2022.

(51) Int. Cl. A61B 17/34

(52) U.S. Cl. CPC A61B 17/3423; A61B 2090/3966

(57) ABSTRACT

Improved systems including rBGCs improved by means of stiffness; working zones and optimized transition zones can traverse the aortic arch into the common carotid and vertebral arteries from a radial approach including navigation of tortuous and individuated anatomical challenges. Mechanical Thrombectomy improved by balloon occlusion prevents thrombus showers and follows 10 years of interventional cardiology and diagnostic use of improved radial approach (TRA) with better systems, making trans-femoral (TFA)less safe and effective than this revised treatment paradigm, unexpectedly better than literature predicted in early parts of the decade. Likewise, carotid stenting may be performed during these intracranial arterial occlusion procedures without swapping out guide catheters as required by the prior art.

### CROSS-REFERENCE TO RELATED APPLICATIONS

This document claims the benefit of priority to U.S. Provisional Application Ser. No. 63/351,137, filed Jun. 10, 2022, of which is hereby incorporated by reference in its entirety.

### BACKGROUND

Endovascular interventions, and particularly neurovascular interventional cases have followed diagnostic cerebral angiograms in the use of transradial access (TRA), or radial access, as discussed herein. Unlike transfemoral or femoral access, challenges include most prominently a lack of devices with dimensions and specifications designed specifically for TRA (Joshi, et al., Journal of Neurointervent Surg. 2020; 12: 886-892). It is respectfully proposed that industry has chosen to instead focus on procurement of regulatory approval for both peripheral vascular and neurovascular system usage as opposed to optimized guide catheters for TRA specifically, targeting the brain. Delivery systems are known for endovascular applications, including therapies and both targeted and systemic treatments, ranging from flow diverters to braided stenting means. Artisans realize that access for devices to treat humans need better ways to initialize and release those interventions. Cerebrovascular disease (stroke) is a major cause of morbidity and the second leading cause of death mortality worldwide. In the U.S. there are 800,000 new or recurrent cases of stroke annually. Sequala of stroke not only impact individual patients but families and society in general secondary to the financial impact, which is multifactorial but includes loss of income, earning potential, productivity, work force supply and loss of time and resources by family to take care of post stroke patients. Post stroke rehabilitation cost alone are more than $50 billion annually in the U.S. alone.

### OBJECTS & SUMMARY OF THE INVENTION

Briefly stated, systems are offered for consideration with optimized transition in balloon guide catheters for TRA particularly (although useful for TFA, etc.), including bridging aortic arch challenges.

According to embodiments, there are provided specialized and optimized transition zone housing balloon guide catheters (BGC) for use by neurointerventionalists.

According to embodiments, BGCs are provided leveraging the First-Pass Effect (FPE) to deliver better clinical outcomes.

According to embodiments, BGCs with enhancements are more likely to be associated with improved recanalization and favorable outcomes.

According to embodiments, BGCs are provided which demonstrate improved clinical outcomes and better reperfusion rates in patients, including shorter reperfusion times.

According to embodiments, improved BGCs are provided which clinical usage of results in lower mortality rates.

With advances in the field of endovascular neurosurgery (neurointerventional surgery) over the last 7 years significant improvement has been made in treatment of cerebrovascular disease, especially acute ischemic stroke (AIS) that have dramatically improved functional outcomes in patients presenting with cerebrovascular disease. Numerous multicenter randomized control trials have now shown the significant benefits of endovascular mechanical thrombectomy (MT) in patient presenting with acute ischemic stroke from large vessel occlusion (LVO). The annual incidence of LVO in the United States as ≈24 per 100,000 population, which translates to an absolute annual incidence of about 80,000 cases. Other studies have shown that as many as 200,000 Americans annually may be eligible for mechanical thrombectomy. Mechanical thrombectomy estimates for 2021 are 39,164 procedures in the US with predicted an annual growth rate of approximately 5-10% over the next decade.

Endovascular treatment (EVT) is the recommended standard of care for treatment of acute LVO in the setting of ischemic stroke as described by multiple stroke and interventional societies worldwide including the American Heart Association (AHA) and American Stroke Association (ASA). The Guidelines for the early management of patients with acute ischemic stroke released in 2019 by the AHA/ASA recommends mechanical thrombectomy for patients presenting with AIS that meet criteria with a stent retriever device or aspiration thrombectomy device. The AHA/ASA guideline also note that the use of a balloon guide catheter (BGC) during mechanical thrombectomy is beneficial. Studies have shown that use of BGC during mechanical thrombectomy improves first-pass effect, results in higher recanalization rate, decreases procedural time, increases NIH stroke scale improvement from admission to 24 hours, lowers mortality rate and improves clinical outcomes at 3 months. Endovascular MT devices have historically been designed for use via a classic transfemoral artery approach (TFA), this is also true of BGC currently available for neurointerventional use.

Fortunately, in recent years the field of endovascular neurosurgery (neurointerventional surgery) has started to adopt a safer and more patient centric transradial artery approach (TRA) for neurointerventional procedures. Transradial access (TRA) has several distinct advantages over traditional transfemoral access (TFA) especially related to patient safety (lower complication rates). More than 10 years of experience reported by interventional cardiologists has demonstrated a reduction in the incidence of hemorrhagic access site complications with TRA compared with TFA. Access site complications from TFA include hematomas, vessel dissection, pseudoaneurysms, embolic complications, and critical limb ischemia. According to data from mechanical thrombectomy trials access site complication rates from TFA are not insignificant, given reported severe access-related adverse events occurring in 2% to 12% of interventions in these trials, with overall access-site complication rate in RCTs for mechanical thrombectomy being 5.13%.

Additional benefits of a TRA as compared to a TFA is ability for early mobilization of patient post procedure given post procedure bedrest is not required for TRA thus facilitating early ambulation, ability to work with physical therapy earlier and allowing for earlier discharge and shorter hospitalization as compared to TFA. Interventional cardiology literature has also demonstrated compared to the TFA, the TRA is significantly reduced median length of stay, improved quality of life (comfort) for the patients after the procedure (measures of bodily pain, back pain, and walking ability). TRA approach has also been shown to decrease healthcare cost compared to TFA in multiple ways: 1. Decreased complications management cost. 2. No need for use of femoral closure device. 3. Decreased level of postop procedure nursing care needed to manage patient given short interval femoral site check for potentially large hematomas are not needed.

There is a rapidly increasing number of neurointerventional surgeons that are transitioning to use of transradial approach for procedures because of the significant benefits. Currently, however, BGC designs are optimized for a femoral approach and cannot be safely, reliably, or easily be used for a transradial approach hence limiting the use for treatment of patients presenting with LVO in setting of MS. Although studies have shown similar results from TRA and TFA for mechanical thrombectomy for LVO many neurointerventionalist including those using TRA for non-mechanical thrombectomy procedures are reluctant because of lack of TRA designed thrombectomy systems. However, despite that lack of appropriately designed TRA systems given the benefits of the transradial approach many interventionalist have started to use TRA for thrombectomies with great success and data is becoming more convincing that the mass transition to TRA adoption is not far away in horizon. Recent meta-analysis of thrombectomies performed via TRA vs TFA revealed that the TRA is a safe alternative to TFA thrombectomies in AIS: with significantly decreased access-site complications and hemorrhagic transformation compared to TFA and comparable first pass effect and puncture to reperfusion times. Another recent study, comparing TRA vs TFA thrombectomy showed that TRA thrombectomy is as fast (time from CT scan to reperfusion) and efficacious (mRS scores 0-2 at 90-days, first-pass effect recanalization of vessel and final TICI 2B-3 reperfusion) as TFA but importantly TRA thrombectomy is significantly safer (6.5% major access site complications in TFA group vs 0% in TRA group).

The improved safety prolife related to access site complications of TRA vs TFA is even more important and relevant in the population suffer from acute stroke. Acute stroke patients often are taking antiplatelet or anticoagulation medications, with many also receiving intravenous thrombolysis mediation (IV tPA) when they present to the emergency room hence a femoral site hematoma and retroperitoneal hematomas can be significantly more complicated and even life threatening. A transradial approach completely negates these potentially life-threatening complications of a transfemoral approach.

Currently available BGC on the market that were designed for a TFA cannot safely and reliably be used on the majority of patients via a TRA because of their design limitations and size. The trajectories via a TRA approach into the cervical vasculature are difficult to navigate using currently available BGC and pose risk of injury to the vasculature and risk of catheter entrapment. However, with appropriate design changes and improvements a BGC can be designed to combine all of the benefits of using a BGC during thrombectomy with the advantages of using a transradial approach.

A radial balloon guide catheter (rBGC) with outer and inner hydrophilic coating with specific transition zone of strength to allow navigation via a transradial approach. Distal zone for atraumatic catheter position within the vessel. A working zone for improved trackability and navigation of the rBGC. A transition zone of strength to decrease incidence of herniation of the catheter into the aortic arch as well as improved kink resistance when transitioning from aortic arch into the cervical vasculature. A support zone to provide stability of the catheter to allow for access, ease of tracking and support via transradial approach. The hydrophilic coating outside will prevent radial artery spasm and provides a lubricious outer surface for catheter advancement in the vasculature. The hydrophilic inner coating will allow smooth wire, catheter and wire exchange and manipulation inside the radial balloon guide catheter. The specifically positioned transition zone will optimize the trajectory of forces needed to position the catheter via a radial approach. These transition zones will be reenforced to ensure the rBGC does not herniate into the aorta during neuro-interventional procedures. The trajectories via a radial approach are significantly different compared to the femoral approach hence currently available BGC cannot navigate into position to be safely, reliably or easily used via a TRA currently. A compliant balloon is mounted near the distal end to provide temporary vascular occlusion during procedures. The balloon catheter also incorporates radiopaque markers to facilitate fluoroscopic visualization and indication of the balloon position. The rBGC system is designed to fit inside commercially available thin-walled 7-French radial sheaths hence significantly decreasing chance of radial artery spasm or occlusion which can occur with bigger BGC systems (requiring 8-French sheath or larger). The rBGC being compatible with 7-French thin wall sheath also obviates the need to use a “sheathless” method for BGC insertion which has been shown to increase risk for injury to radial artery, catheter entrapment and increase complication rates. The inner diameter of the rBGC is compatible with widely available carotid stenting systems and angioplasty balloons that are needed as adjuvant therapies in patients presenting with acute strokes secondary to ruptured carotid artery bifurcation plaques. The rBGC compatibility with commercially available carotid stenting system nullifies the needs to switch out guide catheters for patients presenting with tandem lesions (carotid stenosis and intracranial arterial occlusion).

### BRIEF DESCRIPTION OF THE FIGURES

Various preferred embodiments are described herein with references to the drawings in which merely illustrative views are offered for consideration, whereby:

FIG. 1 shows a schematic of an rBGC according to the present invention, from distal tip to balloon aspiration and injection lumen ports.

FIG. 2 shows a detailed schematic of the instant invention extending past the aortic arch into the neurovascular anatomy, as improved with teachings of the present invention.

In FIG. 3, a detailed view is shown with the balloon guide inflated is show in the upper right corner.

FIG. 4 shows a series of preferred transition zone and variable balloon positions according to the rBGC of the instant teachings.

FIG. 5 shows an rBGC with varying Working Zone lengths which depends on the vessel being treated, patient's vessel anatomy and site of pathology being addressed.

FIG. 6A shows an rBGC with specific segments allowing for flexibility, according to the instant teachings; while,

FIG. 6B illustrates a cross-sectional view according to the present invention;

FIG. 7 shows one example of the optimized transition zone of an rBGC with attached balloon, and,

FIG. 8A shows an example of the rBGC of the present invention with attached balloon;

FIG. 8B shows the tip according to the present invention; and

FIG. 8C illustrates a cross-sectional view according to the present invention.

Corresponding reference characters indicate corresponding components throughout the several views of the drawings, as possible and where technically consistent enough to preserve the accuracy of these schematics and cartooned illustrations. Skilled artisans will appreciate that elements in the figures are illustrated for simplicity and clarity and have not necessarily been drawn to scale. For example, the dimensions of some of the elements in the figures may be exaggerated relative to other elements to help to improve understanding of various embodiments of the present invention. Also, common but well-understood elements that are useful or necessary in a commercially feasible embodiment are often not depicted in order to facilitate a less obstructed view of these various embodiments of the present invention.

### DETAILED DESCRIPTION OF THE INVENTIONS

It is respectfully proposed that the present inventions provide medical devices that have heretofore not been routinely used for and presently available to treat the brain, large vessel occlusions (LVO) and disease states addressed by—for example—spanning the supra-aortic arch, with reliability, trackability and the ability to deliver therapies. This rapidly evolving and important treatment segment has previously been reserved for select highly skilled and experienced practitioners, and direct and required acceptance of this procedure is respectfully believed to advance the progress in science and the useful arts by getting better therapies to the market and advancing the noble goals of global healthcare equity. The present inventor presents novel enhanced rBGC systems, methodologies, processes and products. It is respectfully proposed that both clinical practice and the literature support TRA's features and benefits ranging from 10 years of cardiologist data showing reduction in hemorrhagic access site complications (adv. TFA); early mobilization of patients without compromised mobility, and concomitant early discharge and lower hospitalization expense. However, the need to address LVO presenting patients requires, for example, an improved rBGC of the present invention to avoid pitfalls associated with trying to fix the round BGC for TRA into a square hole of TFA; namely, safety, reliability and ease of use issues abound.

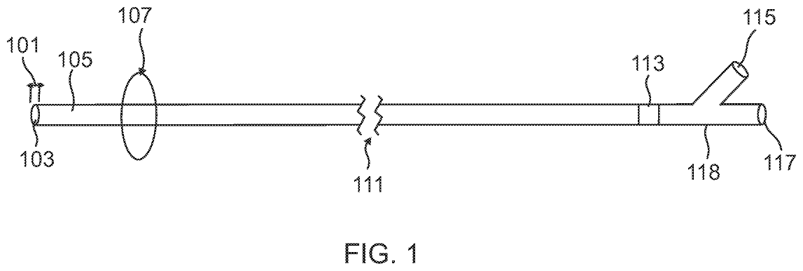

Referring now to FIG. 1 of the schematic cartooned line drawings offered for consideration merely for illustrative, as opposed to limiting, purposes, shown is a core element of the radial balloon guide catheter (rBGC) of the present invention. It is further respectfully submitted that with regard to this illustration and those of the eight figures, and subparts labeled for ease of use with alpha-numeric letters, that details known to those of skill in the art have been omitted for clarity of presentment. Likewise, artisans are well aware that the Seldinger technique, and numerous standard elements supporting the same from guidewires to trocars, needles, etc. which are not needed to be further discussed, with reference to endovascular and/or brain, LVO and/or peripherals vascular access techniques U.S. Pat. Nos. 8,088,140; 8,070,791; 8,197,493; 8,545,514; 8,585713; 10,258,774; and 10,724,511, are expressly incorporated by reference, as if fully set forth, and shall be disclosed along with at least about 8 references within a compliant Information Disclosure Statement (IDS) to be filed herewith. FIG. 1 shows a device proposed as core component of the instant system, namely distal tip 101 positioned at the distal end of this device adjacent to marker 103 at the edge of working zone 105. Working zone 105 likewise features compliant balloon 107, transition zone 111, protector section 113, Balloon aspiration port 115, connector 118 and injection port 117.

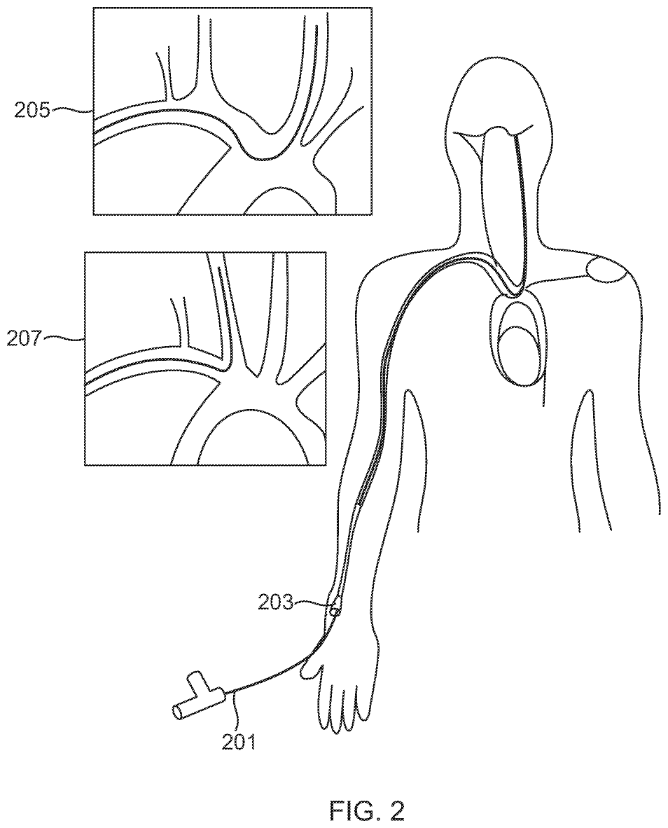

Referring now also to FIG. 2 and to FIG. 3, artisans understand readily that the medical device system, as defined herein and claimed below as new novel and non-obvious may be used for both Trans-radial (TRA) and Trans-femoral (TFA) access, along with any other peripheral access sites, for various targets. For ease of illustration only, the example case being shown is radial, similarly to the device being styled a “radial balloon guide catheter”—those skilled in the art know this to mean each of these terms and semantic variations are defined within this specification and claimed interchangeably. In other words, the novelty of the instant system is shown by radial but may be adapted for any other surgical use case, and along with the Seldinger technique, and any and all known endovascular, peripheral vascular, percutaneous and even open surgical related practices known to artisans or with the same “function/way/result” tests for any scope of equivalents. FIG. 2. Shows this first example of how the instant teachings, as qualified above and claimed below are known to work with patients. Here as further defined in step-by-step detail below, this example shows an example radial balloon guide system 201 being advanced via radial sheath 203 with standard endovascular techniques (as described in detail below) to [referring also to the detailed boxes superposed herein] to employ the mechanically optimized transition zones to span the surpa-aortic arch, and access select main supra-aortic branches here the left CCA 205, and/or right CCA 207. FIG. 3 likewise provides views of the instant system including radial balloon guide system 301 via radial sheath 303 showing the balloon guide inflated in the brain, enabling the Physician/Interventionalist to address any disease state targeted, mostly intracranial arterial occlusions, inter alia. Again, without needing to provide details known to those of skill in the art, a conventional carotid stenting system may be paired with the instant system to allow for the treatment of tandem occlusions with the same novel rBGC, at this point deploying any known carotid stenting system or assemblies while targeting the intracranial arterial occlusion shown with rBGC inflated at 308.

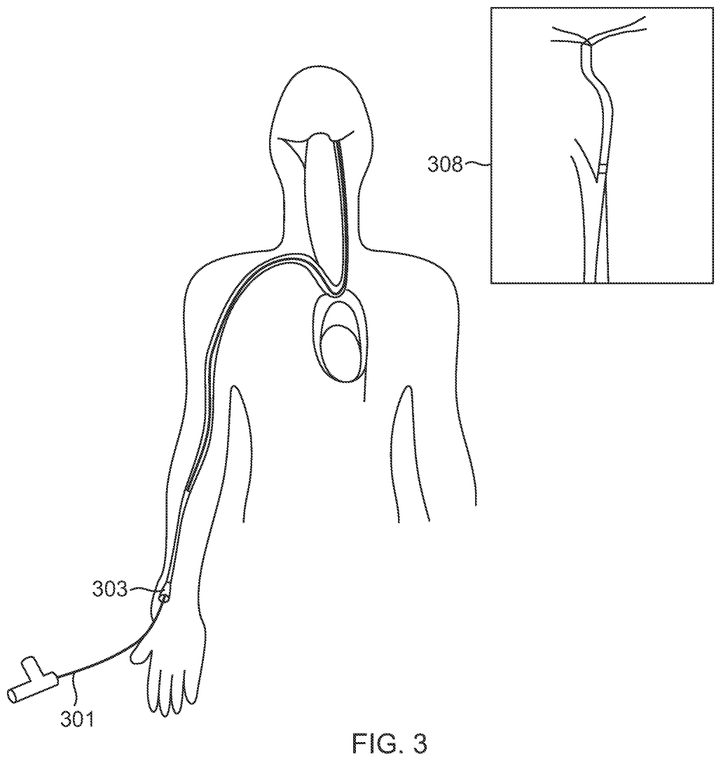

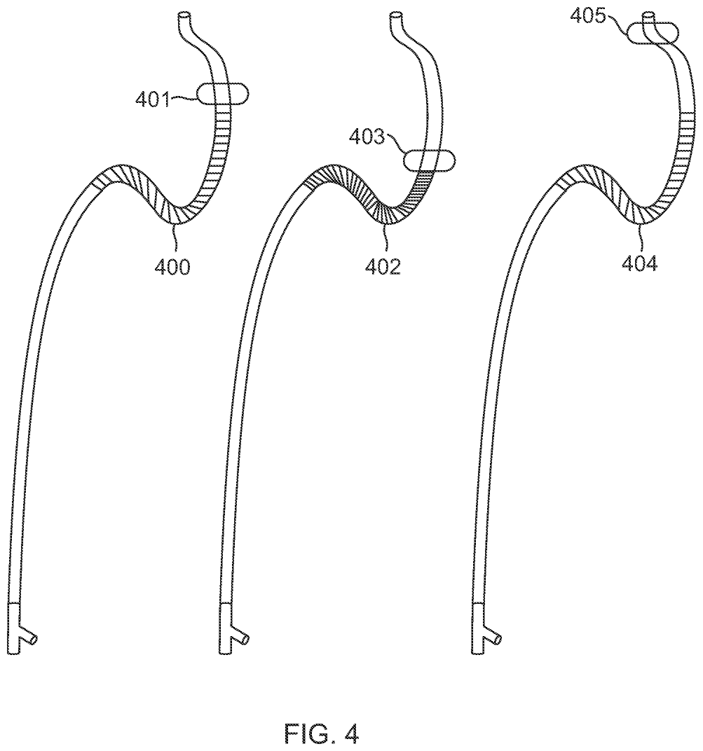

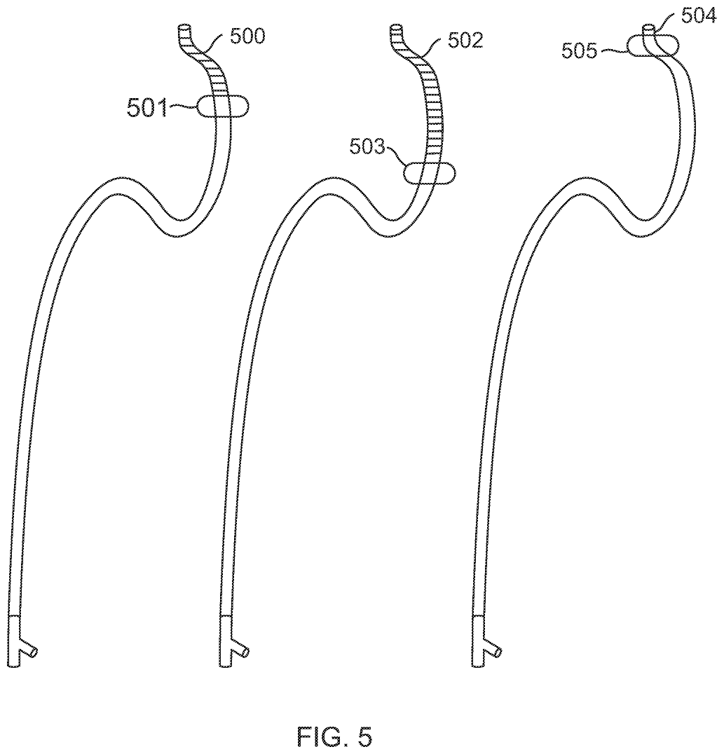

Referring now to FIG. 4 and FIG. 5, there are shown a plurality of distinct configurations for transition zones 400, 402, 404 and their concomitant balloon positions 401, 403 and 405. The present inventor likewise configures working zones 500, 502 and 504 to accommodate various balloon positions 501, 503 and 505.

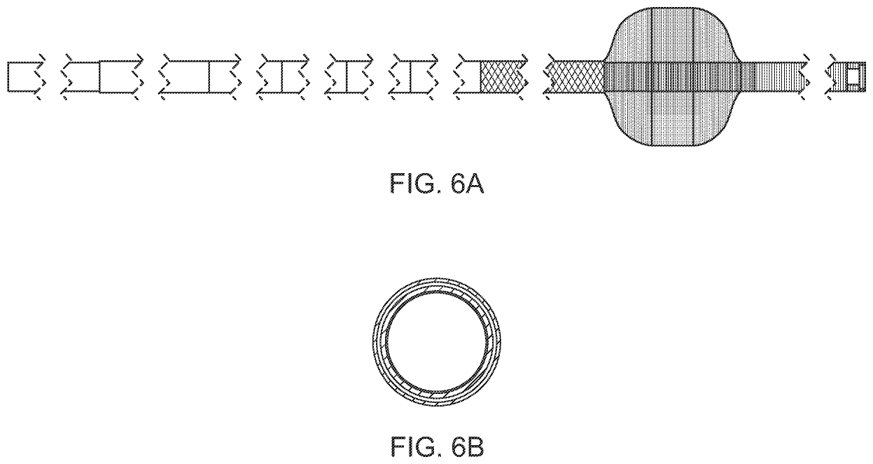

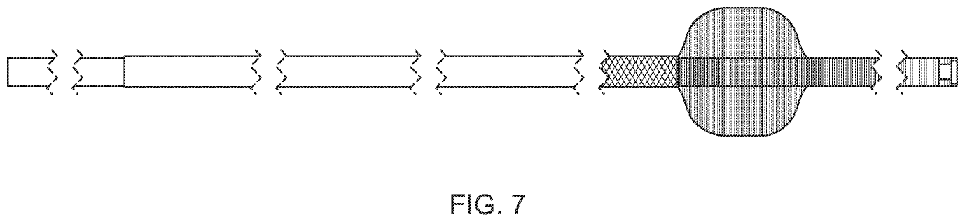

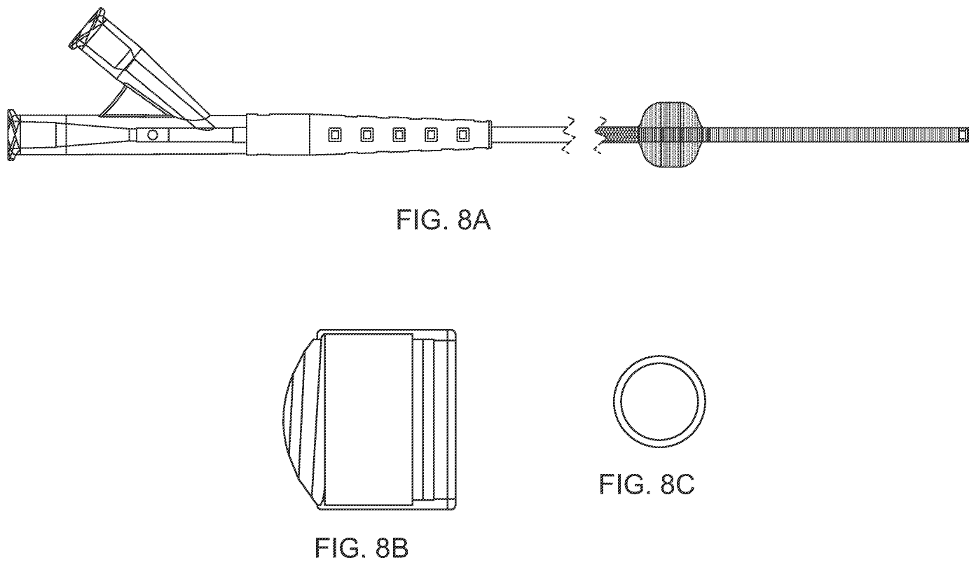

Finally, referring to FIG. 6A, 6B, FIG. 7 and FIGS. 8A, 8B and 8C, unexpected results of variation of the stiffness values, transition zones and working zones have resulted in points of novelty of the present invention, it is respectfully submitted. For example, alternating PEBAX and NUESOFT brands of materials enables the present invention to be customized to anatomy of lesions to be treated and configurations of different transition zones, and working zones tailored to the anatomy to be treated, as shown in prior figures, along with balloon positions. FIG. 6A shows distal end of an example intravascular catheter assembly featuring working lumen which attaches to inflation lumen, and in this example consists essentially of PEBAX 72, with the next segment being PEBAX 63D, then PEBAX 55D followed by PEBAX PEBAX 35D then PEBAX 253/8/23, respectively. The example balloon is NEUSOFT 842A, with the proximal end being NEUSOFT 62A in this example. FIG. 6B illustrates the relative positions of inner lumens (working) and outer lumens (inflation lumens). In FIG. 7 there is shown the coated subassembly of the example intravascular catheter, while FIG. 8A shows detail on the proximate catheter end, 8B how the polymer tip goes past the liner to create an atraumatic tip, and 8C the relative maxima and minima for the working zones. Desired stiffness values have included use at least one material, selected from the group consisting essentially of: Grilamid L25; Pebax 72D, Pebax 63D, Pebax 55D, Pebax 45D, Pebax 35D, Pebax 25D, Neusoft 42A, Neusoft 862A, Neusoft 852A, andNeusoft 842A. By variating these features, various anatomies and lesion sites can be addressed to span supra-aortic challenges as heretofore unavailable on the market or for pre-clinical usage.

Recited below is the inventor's optimal technique for use of the instant disclosures, for one application of many. Those skilled in the art understand that both on and off label usages of the present inventions are roughly analogous to uses of the (neither approved for this indication nor customized to this application) Flowgate™ brands of medical devices, namely, as indicated for use in facilitating the insertion and guidance of the intravascular catheter into a selected blood vessel in the peripheral and neurovascular systems. The balloon provides temporary occlusion during these, and other merely diagnostic generally angiographic procedures. The balloon is also indicated for use as a conduit. Using standard endovascular technique, a thin walled (radial specific) sheath is inserted into the radial artery after a “radial cocktail” (as per published literature) is infused and connected to heparin drip.

Next, the Radial Balloon Guide Catheter (rBGC) is navigated tri-axially over a long (≥130 cm) access catheter of choice (Vert, Berenstein, VTK, Sim, brands of medical devices, which are commercially available) over a 0.035 inch or 0.038 inch glide wire into the aortic arch. Next the access catheter is used to select the vessel origin of interest (common carotid or vertebral artery). Under fluoroscopic guidance the wire is navigated up the vessel of interest followed by navigation of the access catheter and then the rBGC until positioned in place for intervention.

Next the wire and access catheter are removed and rBGC in left in place in the vessel of interest and connected to a heparin drip as per protocol.

Next if the patient has a tandem lesion (severe carotid stenosis in addition to the blockage in the intracranial vessels) then carotid stenting can be performed using commercially available carotid stenting system in standard fashion at the discretion of the neurointerventionalist prior to or after intracranial mechanical thrombectomy is performed (as described below).

Next the endovascular thrombectomy device of choice is navigated inside the rBGC to perform thrombectomy. Balloon on rBGC is inflated with 50% contrast and 50% saline to arrest flow during thrombus removal via the balloon aspiration/inflation port. Once thrombus is removed, aspiration is applied to the Main Lumen of the rBGC to remove and debris from the thrombectomy hence preventing distal emboli.

Next the balloon is deflated by applying aspiration suction via the balloon aspiration/inspiration port.

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary, and that the actual parameters, dimensions, materials, and/or configurations will depend upon the specific application or applications for which the teachings of the present disclosure is/are used.

Those skilled in the art will recognize or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

The indefinite articles “a” and “an,” as used herein in the specification and in the claims, unless clearly indicated to the contrary, should be understood to mean “at least one.” The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified, unless clearly indicated to the contrary.

The terms and expressions which have been employed herein are used as terms of description and not of limitation, and there is no intention, in the use of such terms and expressions, of excluding any equivalents of the features shown and described (or portions thereof), and it is recognized that various modifications are possible within the scope of the claims. Accordingly, the claims are intended to cover all such equivalents.

Furthermore, the described features, structures, or characteristics of the invention may be combined in any suitable manner in one or more embodiments. In the following description, numerous specific details are provided to provide a thorough understanding of embodiments of the invention. One skilled in the relevant art will recognize, however, that the invention may be practiced without one or more of the specific details, or with other methods, components, materials, and so forth. In other instances, well-known structures, materials, and so forth. In other instances, well-known structures, materials, or operations are not shown or described in detail to avoid obscuring aspects of the invention.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention, and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

1. A system for radial access to, and ingress through the supra-aortic arch to select supra-aortic branches, which comprises, in combination:
an intravascular catheter having a proximate and a distal end, a compliant balloon positioned closer to the distal end of the catheter between a working zone and a transition zone along said intravascular catheter;
a protector section, a connector, a balloon aspiration port, an injection lumen port; and
radiopaque markers likewise functionally connected upon the intravascular catheter.

2. The system of claim 1, further comprising
an access catheter, of at least 120 cm;
a glide wire dimensioned between 0.035 and 0.038 inches in diameter; and,
a radial sheath being able to house the system being approximately 7 Fr.

3. The system of claim 2, wherein the working zones and
optimized transitional zones leverage variable balloon positioning and balloon guide inflation for spanning the supra-aortic arch and supporting roughly orthogonal targeted vectors within the supra-aortic arches to address intracranial arterial occlusions.

4. The system of claim 3, further comprising the ability to address tandem lesions within the carotid artery by using the same catheter to deploy conventional carotid stenting systems by advancing the same through an injection/inner lumen established to address intracranial arterial occlusions.

5. An improved radial balloon guide catheter (rBGC), having a length spanning from a distal tip to a proximal series of ports for lumen injection and balloon aspiration, which comprises, in combination:
a compliant balloon;
at least an inner and outer hydrophilic coating on the length of the catheter;
a plurality of specific transition zones of strength located between the compliant balloon and the proximal end of the catheter;
whereby said specific transition zones serve to optimize the trajectory of forces needed to position the catheter via a radial approach.

6. The improved rBGC of claim 5, further comprising:
the (rBGC) allowing for smooth catheter, wire exchange and manipulation, inside the rBGC owing to said hydrophilic inner coating.

7. The improved rBGC of claim 6, where said optimized transition zone is reinforced to ensure the rBGC does not herniate into the aorta during neurointerventional, mechanical thrombectomy, carotid stenting and other vascular or cerebrovascular surgical procedures.

8. The improved rBGC of claim 7, further comprising:
a soft distal tip having radiopaque markers, which minimizes chances of vessel insult and injury.

9. The improved rBGC of claim 8, further comprising:
at least a working zone distal to a balloon being soft enough to conform to vessel turns and tortuosity, enabling ingress to the supra-aortic arches and target vessels above the same.

10. The improved rBGC of claim 9, further comprising a segment with a balloon which balloons when dilated, shall temporarily arrest blood flow during thrombectomy to prevent distal emboli; and once embolus is removed, aspiration is applied to injection/main lumen to remove any embolic debris distal to balloon secondary to thrombectomy.

11. The improved rBGC of claim 10, being effective for aspirating a balloon by via a balloon aspiration port to deflate said balloon to resist anterograde blood flow post-thrombectomy.

12. A process for using an improved rBGC for treating intracranial arterial occlusions, which comprises at least the steps of:
providing a thin walled radial sheath housing an intravascular rBGC;
inserting the sheath into the radial artery following infusion of a predetermined anti-clotting, anti-inflammatory and anti-vasospasm mixture into the patient subcutaneously, and connecting the patient to a heparin-drip;
navigating the rBGC triaxially over an access catheter, being longer than at least about 120 cm, over a wire being between at least about 0.035 and 0.038 in diameter into position within the supra-aortic arch;
using the access catheter to select at least one vessel from the group of supra-aortic main branches consisting essentially of the Common Carotid Artery (CCA) and the Vertebral Artery (VA);
navigating the wire up the vessel of interest under fluoroscopic guidance, followed by the access catheter and then the rBGC until positioned in place for intervention;
removing the wire and access catheter leaving the rBGC in place in the vessel of interest;
navigating any endovascular thrombectomy device inside the rBGC to perform thrombectomy;
inflating the balloon on the rBGC with 50% contrast and 50% saline to arrest flow during thrombus removal via balloon aspiration/inspiration port;
applying aspiration to the main lumen of the rBGC once thrombus removed to remove any other debris from the thrombectomy preventing distal emboli; and
deflating the balloon by application of aspiration/suction via the balloon aspiration/inspiration port

13. The process of claim 12, wherein the step of removing the wire and access catheter is followed by a step of treating a tandem lesion within the Carotid Artery (carotid bifurcation usually)[CA] by emplacing any known carotid stenting system through the access pathway and rBGC to deliver therapy, without changing the guide catheter.

14. The process of claim 13, the diameter of the rBGC system being
inserted into the thin walled radial sheath and navigated retrograde into the aortic arch and up at least one cervical vessel selected from the group of either CCA and VA being at least about 5 Fr.

15. The process of claim 14, wherein the reinforced and optimized transition zones enable navigation through tortuous anatomy defined by the angle of vessels relative to the aortic arch as approached in transradial access.

16. The process of claim 15, said segments of the catheter being emplaced within significant anatomical tortuosity and effective for the same, based upon reinforced catheter segments preventing catheter kinking, herniation and kick-out.

17. The process of claim 16, wherein the step of treating a tandem lesion further comprises rBGC positioning within carotid arteries with balloon inflated, whereby distal catheter portions introduce any MT devices while said balloon being inflated, provide temporary flow arrest to prevent emboli.

18. The process of claim 17, further comprising a plurality of differently reinforced transition zones at varying sections along said catheter driven by catheter length and patient's anatomy, site of pathology and subject vessel being treated.

19. The system of claim 1, the intravascular catheter further comprising a plurality of segments of different materials having different stiffness values selected from the group consisting essentially of: GRILAMID L25, PEBAX PEBAX 45D, NEUSOFT 862A, PEBAX35D, PEBAX 55D, PEBAX 63D, PEBAX 72D, NEUSOFT 852A and NEUSOFT 842A.

20. The system of claim 1, the balloon having a maximum length of 10 mm.
